(19) United States
(12) Patent Application Publication
(10) Pub. No.: US 2024/0099566 A1
(43) Pub. Date: Mar. 28, 2024

(54) MEDICAL DEVICE COMPONENTS, ASSEMBLIES, AND ASSOCIATED METHODS

(71) Applicant: Boston Scientific Scimed, Inc., Maple Grove, MN (US)

(72) Inventors: Man Minh NGUYEN, Harvard, MA (US); Elizabeth MARK, Boston, MA (US)

(73) Assignee: Boston Scientific Scimed, Inc., Maple Grove, MN (US)

(21) Appl. No.: 18/471,501

(22) Filed: Sep. 21, 2023

Related U.S. Application Data
(60) Provisional application No. 63/376,679, filed on Sep. 22, 2022.

(51) Int. Cl. A61B 1/008; A61B 1/005

(52) U.S. Cl. CPC A61B 1/008; A61B 1/0057; A61B 1/273

(57) ABSTRACT

An elevator of a medical device may comprise: a plurality of segments joined together by at least one hinge, and a control element coupled to at least one segment of the plurality of segments. A first segment of the plurality of segments may be a discrete structure. Proximal or distal movement of the control element may be configured to move the at least one segment of the plurality of segments relative to another of the plurality of segments.

### CROSS-REFERENCE TO RELATED APPLICATIONS

This application claims the benefit of priority under 35 U.S.C. § 119 from U.S. Provisional Application No. 63/376,679, filed Sep. 22, 2022, which is incorporated by reference herein in its entirety.

### TECHNICAL FIELD

Various aspects of this disclosure relate generally to medical device components, assemblies, and associated methods. In particular, aspects of this disclosure pertain to elevators of duodenoscopes, among other aspects.

### BACKGROUND

Duodenoscopes may include a handle and a sheath, and the sheath may be insertable into a body lumen of a subject. The sheath may terminate in a distal tip portion, which may include features such as optical elements (e.g., camera, lighting), air/water outlets, and working channel openings. An elevator may be disposed at a distal tip and may be actuatable in order to change an orientation of a medical device/tool passed through the working channel. For example, the elevator may be pivotable or otherwise movable.

Elements in the handle may control the elements of the distal tip. For example, buttons, knobs, levers, etc. may control elements of the distal tip. The elevator may be controlled via a control mechanism in a handle, such as a lever, which may be attached to a control wire that attaches to the elevator. When an actuator (e.g., a lever) is actuated, the wire may move proximally and/or distally, thereby raising and/or lowering the elevator.

### SUMMARY

Each of the aspects disclosed herein may include one or more of the features described in connection with any of the other disclosed aspects.

In an example, an elevator of a medical device may comprise: a plurality of segments joined together by at least one hinge, and a control element coupled to at least one segment of the plurality of segments. A first segment of the plurality of segments may be a discrete structure. Proximal or distal movement of the control element may be configured to move the at least one segment of the plurality of segments relative to another of the plurality of segments.

Any of the devices disclosed herein may include any of the following features, additionally or alternatively, in any combination. All of the plurality of segments may be discrete structures. The plurality of segments may consist of the first segment and a second segment. In a first configuration, all of the plurality of segments may lie along an approximately straight line. The approximately straight line may be approximately parallel to a longitudinal axis of the medical device. The control element may be first control element, and the elevator may further comprise a second control element coupled to the at least one segment of the plurality of segments. Proximal or distal movement of the second control element may be configured to move the at least one segment of the plurality of segments relative to another of the plurality of segments. Each of the plurality of segments may define a first channel for receiving the first control element. Each of the plurality of segments may define a second channel for receiving the second control element. The first control element may be coupled to a first actuator of a handle of the medical device. The second control element may be coupled to a second actuator of a handle of the medical device. The first actuator may be separately actuatable from the second actuator. A distalmost segment of the plurality of segments may include a socket on an outer surface of the distalmost segment. The socket may be configured to receive a distal end of the control element. The first segment may include a first distal surface. A second segment of the plurality of segments may include a first proximal surface and a second distal surface. A third segment of the plurality of segments may include a second proximal surface. The first distal surface of the first segment and the first proximal surface of the second segment may be separated by a first angle. The second distal surface of the second segment and the second proximal surface of the third segment may be separated by a second angle. The second angle may differ from the first angle. Each segment of the plurality of segments may include a base wall, a first side wall, and a second side wall. The first side wall of each segment may define a channel configured to receive the control element. A width of the first side wall of each segment may be greater than a width of the second side wall of each segment. The at least one hinge may include a pin or rivet. A distalmost segment of the plurality of segments may include a distally extending protrusion. The control element may be affixed to the protrusion. A proximalmost segment of the plurality of segments may include an axle configured to be rotatably coupled to a distal tip of the medical device. At least one of the plurality of segments may be configured to rotate, while the control element moves distally, relative to the proximalmost segment of the plurality of segments before the proximalmost segment of the plurality of segments rotates with respect to the distal tip of the medical device.

In another example, an elevator of a medical device may comprise a plurality of segments joined together by at least one hinge; a first control element coupled to at least one segment of the plurality of segments; and a second control element coupled to the at least one segment of the plurality of segments. Proximal or distal movement of the first control element may be configured to move the at least one segment of the plurality of segments relative to another of the plurality of segments, and proximal or distal movement of the second control element may be configured to move the at least one segment of the plurality of segments relative to another of the plurality of segments.

Any of the devices described herein may include any of the following features, additionally or alternatively, in any combination. At least two segments of the plurality of segments are formed monolithically with one another. Each of the plurality of segments may define a first channel for receiving the first control element. Each of the plurality of segments may define a second channel for receiving the second control element.

In another example, an elevator of a medical device may comprise: a first segment; a second segment; a third segment; a first hinge between the first segment and the second segment; and a second hinge between the second segment and the third segment. The first segment may include a first distal surface, the second segment may include a first proximal surface and a second distal surface, the third segment may include a second proximal surface, the first distal surface of the first segment and the first proximal surface of the second segment may be separated by a first angle, wherein the second distal surface of the second segment and the second proximal surface of the third segment may be separated by a second angle, and the second angle may differ from the first angle.

Any of the devices described herein may include any of the following features, additionally or alternatively, in any combination. The first segment, the second segment, and the third segment may be formed monolithically with one another.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIGS. 1A and 1B depict an exemplary medical device.

FIGS. 2A-2C depict an exemplary elevator that may be used with the exemplary medical device of FIGS. 1A-1B.

FIGS. 3A-3C depict another exemplary elevator.

FIGS. 4 and 5 depict aspects of the elevators of FIGS. 2A-3C.

FIGS. 6A-6E depict another exemplary elevator.

FIGS. 7A-7E depict an exemplary elevator in various configurations.

FIGS. 8A-8B depict a further exemplary elevator.

FIGS. 9A-9C depict another exemplary elevator.

FIGS. 10A-10C depict a further exemplary elevator.

### DETAILED DESCRIPTION

A duodenoscope or other medical device (e.g., an endoscopic ultrasonography (“EUS”) scope) may include a distal tip having an elevator. The elevator may be operatively connected to an actuator in a handle of the duodenoscope. When an operator activates the actuator, the elevator may be raised/lowered. When an operator inserts an accessory device (e.g., an instrument tor guidewire) into a working channel of the duodenoscope and advances the instrument through the working channel, the elevator originally may be in a lowered configuration. After the accessory device has been extended out of a distal opening of the working channel, the elevator may be raised so as to deflect a distal tip of the instrument. For example, such deflection may be utilized to allow an operator to access a subject's biliary tract and/or to cannulate a subject's major papilla.

A conventional, rigid elevator with a scoop-shaped guiding surface may deflect an instrument in a lowered configuration, as well as in a raised configuration. In other words, such an elevator may partially articulate/deflect an accessory device even in a fully open/lowered configuration. Such a rigid, scoop-shaped elevator also may allow for limited articulation options (e.g., only a fully open/down position or a fully closed/up position). A segmented elevator may allow for an elevator to fully flatten (e.g., lie approximately parallel to a longitudinal axis of the distal tip of the duodenoscope) when in an open/down position. A fully flattened elevator may allow for extending an accessory device straight out of a distal surface of the duodenoscope (e.g., approximately parallel to the central longitudinal axis of the distal tip of the duodenoscope). A segmented elevator may also or alternatively facilitate incremental deflection of the accessory device. An operator may have additional control over the accessory device because the operator can gradually articulate the elevator, segment by segment, to facilitate access to anatomical structures (e.g., bile and pancreatic ducts). A segmented elevator may allow the operator to deflect, and maintain the deflected position of, the accessory device at a plurality of different angles transverse from a central longitudinal axis of the duodenoscope.

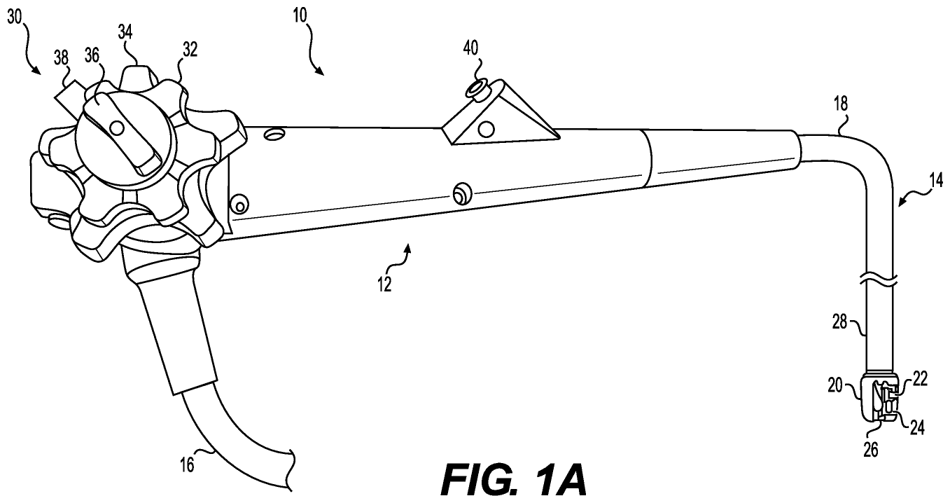

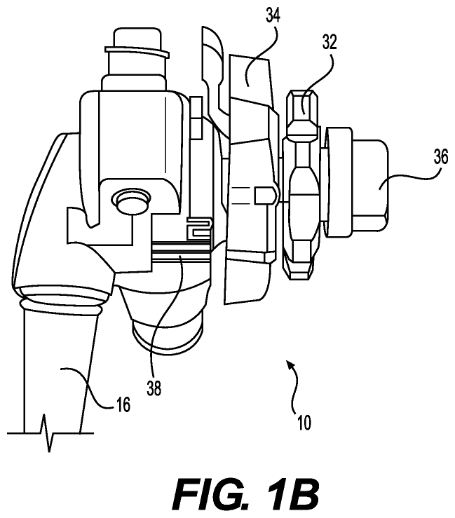

FIG. 1A depicts an exemplary duodenoscope 10 having a handle 12 and an insertion portion 14. FIG. 1B shows a proximal end of handle 12. Duodenoscope 10 may also include an umbilicus 16 for purposes of connecting duodenoscope 10 to sources of, for example, air, water, suction, power, etc., as well as to image processing and/or viewing equipment. Although a duodenoscope may be referenced herein, it will be appreciated that the disclosure also encompasses endoscopes, bronchoscopes, gastroscopes, EUS scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 14 may include a sheath or shaft 18 and a distal tip 20. Distal tip 20 may include an imaging device 22 (e.g., a camera) and a lighting source 24 (e.g., an LED or an optical fiber). Distal tip 20 may be side-facing. That is, imaging device 22 and lighting source 24 may face radially outward, perpendicularly, approximately perpendicularly, or otherwise transverse to a longitudinal axis of shaft 18 and distal tip 20. Additionally or alternatively, distal tip 20 may include one or more imaging devices 22 that face in more than one direction. For example, a first imaging device 22 may face radially outward, and a second imaging device 22 may face distally (approximately parallel to a longitudinal axis of distal tip 20/shaft 18).

Distal tip 20 may also include an elevator 26 for changing an orientation of a tool inserted in a working channel of duodenoscope 10. Elevator 26 may alternatively be referred to as a swing stand, pivot stand, raising base, or any suitable other term. Elevator 26 may be pivotable via, e.g., an actuation wire or another control element that extends from handle 12, through shaft 14, to elevator 26.

A distal portion of shaft 18 that is connected to distal tip 20 may have a steerable section 28. Steerable section 28 may be, for example, an articulation joint. Shaft 18 and steerable section 28 may include a variety of structures which are known or may become known in the art.

Handle 12 may have one or more actuators/control mechanisms 30. Control mechanisms 30 may provide control over steerable section 28 or may allow for provision of air, water, suction, etc. For example, handle 12 may include control knobs 32, 34 for left, right, up, and/or down control of steerable section 28. For example, one of knobs 32, 34 may provide left/right control of steerable section 28, and the other of knobs 32, 34 may provide up/down control of steerable section 28. Handle 12 may further include one or more locking mechanisms 36 (e.g., knobs or levers) for preventing steering and/or braking of steerable section 28 in at least one of an up, down, left, or right direction. Handle 12 may include an elevator control lever 38 (see FIG. 1B). Elevator control lever 38 may raise and/or lower elevator 26, via connection between lever 38 and an actuating wire that extends from lever 38, through shaft 18, to elevator 26. A port 40 may allow passage of a tool through port 40, into a working channel of the duodenoscope 10, through sheath 18, to distal tip 20.

In use, an operator may insert at least a portion of shaft 18 into a body lumen of a subject. Distal tip 20 may be navigated to a procedure site in the body lumen. The operator may insert an accessory device (not shown) into port 40, and pass the accessory device through shaft 18 via a working channel to distal tip 20. The accessory device may exit the working channel at distal tip 20. The user may use elevator control lever 38 to raise elevator 26 and angle the accessory device toward a desired location (e.g., a papilla of the pancreatico-biliary tract). The user may use the accessory device to perform a medical procedure.

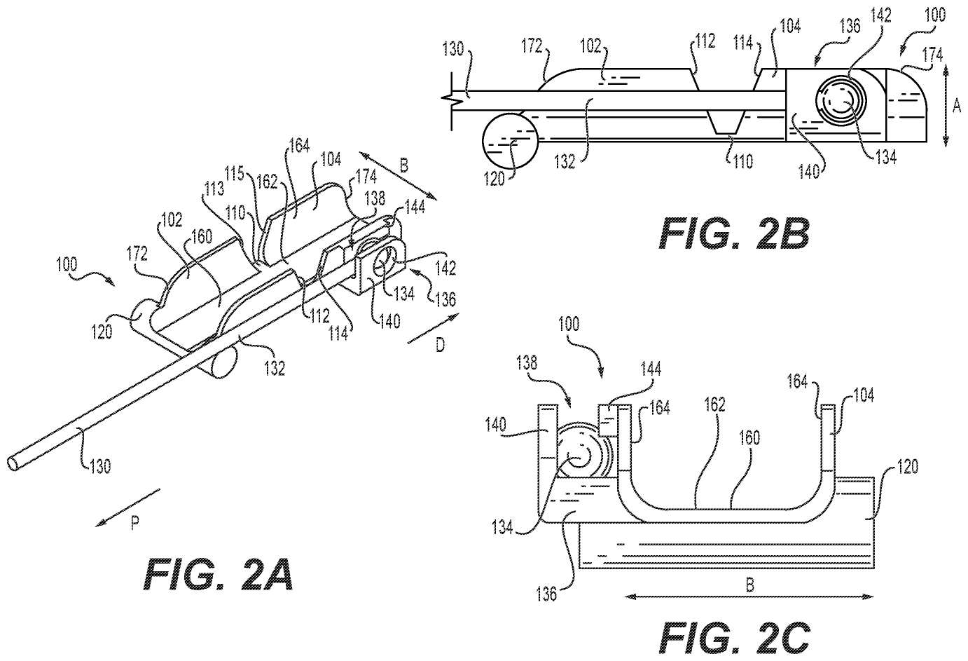

FIGS. 2A-10C depict example elevators and features thereof. Although features may be described with respect to a particular example herein, it will be appreciated that the features described herein may be combined in any suitable combination. The elevators of FIGS. 2A-10C may include a plurality of segments, such that the elevators are flexible along their lengths (i.e., along a longitudinal direction of the disclosed elevators). Any of the elevators described herein may be used in conjunction with duodenoscope 10 or another medical device and may have any of the features of elevator 26. For convenience, features of duodenoscope 10 will be referenced below but will not be understood to limit features of the elevators described herein.

FIGS. 2A-2C depicts a first example elevator 100. FIG. 2A depicts a perspective view of elevator 100. FIG. 2B depicts a side view of elevator 100, and FIG. 2C depicts a proximal-facing view of elevator 100. Elevator 100 may include a first, proximal segment 102 and a second, distal segment 104 (only distal segment 104 is visible in the view of FIG. C). Elevator 100 may include exactly two segments 102, 104. The arrows “P” and “D” in FIG. 2A depict proximal and distal directions. In FIG. 2C, the distal direction extends out of the page, and the proximal direction extends into the page. First segment 102 and second segment 104 may be joined at a hinge 110. A proximal end of first segment 102 (i.e., a proximalmost segment of elevator 100) may include an axle 120. A control element 130 may be coupled to second segment 104.

First segment 102 and second segment 104 may be joined at a hinge 110. Hinge 110 may include, for example, a living hinge. In examples, first segment 102 and second segment 104 may be formed from a single, monolithic piece of material, such as a flexible piece of material. For example, hinge 110 and/or other portions of elevator 100 may include plastic or metal. Materials of elevator 100/hinge 110 may be flexible and may not have shape memory features. Additionally or alternatively, hinge 110 may be overmolded or include other attachment means with plastic or metal wires in order to create a living hinge. In such configurations, first segment 102 and second segment 104 may be formed from separate pieces of material (not be monolithically formed) or may be monolithically formed with additional components overmolded/attached. Elevator 100, including the segments and hinges thereof, may include any suitable materials, such as metal or plastic. Elevator 100 may be formed by any suitable method of manufacturing, including additive or subtractive manufacturing methods such as molding, three-dimensional printing, carving, or other methods.

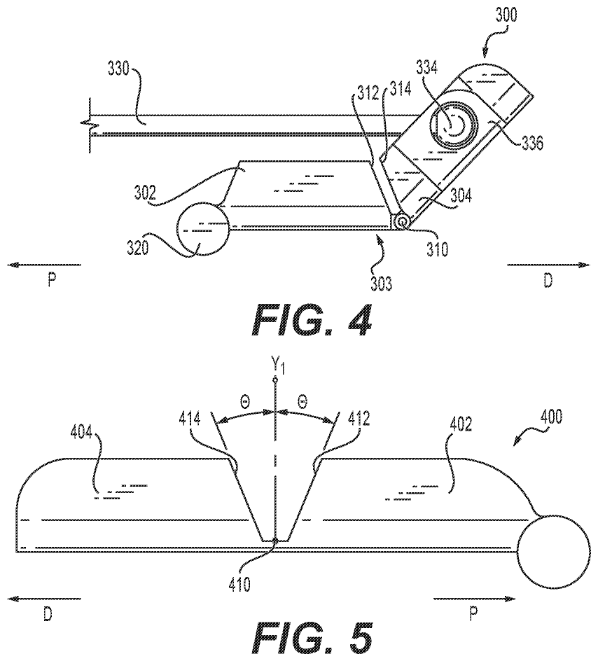

Hinge 110 may have a smaller height (in a direction “A” shown in FIG. 2B, perpendicular to the proximal/distal directions) than first segment 102 and second segment 104. In other words, elevator 100 may be thinner at hinge 110 than at first segment 102 or second segment 104. Elevator 100 may optionally include a recess at hinge 110 (e.g., a one or more of first segment 102 or second segment 104 may include a recess). Thus, elevator 100 may be flexible at hinge 110. Thus, as described in further detail with respect to FIG. 4, below, second segment 104 may be able to articulate (rotate/pivot) with respect to first segment 102 about hinge 110.

A distal surface 112 of first segment 102 and a proximal surface 114 of second segment 104 may taper such that elevator 100 includes a notch defining hinge 110. The notch may have an approximately V-shape. As shown particularly in FIG. 2B, the notch may have a flattened V-shape, with hinge 110 defining a flattened surface of the V-shape. For example, distal surface 112 of first segment 102 may taper such that a height of first segment 102 decreases in a distal direction along an extent of distal surface 112. Proximal surface 114 of second segment 104 may taper such that a height of second segment 104 increases in a distal direction along an extent of proximal surface 114. Hinge 110 may extend between distal surface 112 and proximal surface 114. For example, hinge 110 may include a thin strip of material. Hinge 110 may additionally or alternatively include any properties that are known or become known of a living hinge. When elevator 100 bends about hinge 110 (as described in further detail below), proximal surface 114 of second segment 104 may contact/abut/mate with distal surface 112 of first segment 102. Thus, proximal surface 114 and distal surface 112 may define an articulation/bending limit/range of hinge 110 (i.e., bending may be limited by interaction of proximal surface 114 and distal surface 112, and an articulated profile of elevator 100 may be at least partially defined by the interaction between proximal surface 114 and distal surface 112). In some examples, first segment 102 may include a pair of distal surfaces 112, 113 (one on each side of first segment 102) and second segment 104 may include a pair of proximal surfaces (one on each side of second segment 104) 114, 115. Distal surface 112 may have substantially the same shape as distal surface 113, proximal surface 114 may have substantially the same shape as proximal surface 115, and proximal surface 115 may be configured to abut distal surface 113 when proximal surface 114 abuts distal surface 112.

An axle 120 may be disposed at a proximal end of first segment 102. In some examples, axle 120 may be formed monolithically with first segment 102. Axle 120 may have a round cross-section, as shown in FIG. 2B. As shown in FIGS. 2A and 2C, axle 120 may extend beyond first segment 102 along a direction B (perpendicular to direction A and the proximal/distal directions), on either side of first segment 102. In alternatives, axle 120 may extend beyond first segment 102 along direction B on only one side of first segment 102 or may not extend beyond first segment 102 along direction B. Axle 120 may be configured to interact with features of distal tip 20 to allow elevator 100 to be rotatable relative to other elements of distal tip 20. For example, distal tip 20 may include complementary features for receiving axle 120. Axle 120 may be rotatable along an axis that is approximately parallel with the B direction but may otherwise be substantially unable to move relative to distal tip 20.

Control element 130 may be coupled to second segment 104. Control element 130 may include, for example, wire(s), cable(s), rod(s), chain(s), string(s), cord(s), or other suitable structures. For example, as shown in FIGS. 2A-2C, control element 130 may include a wire 132 and a protrusion 134 at a distal end of wire 132. For example, protrusion 134 may include a ball. Alternatively, protrusion 134 may have other shapes (e.g., cylindrical, flat like a head of a nail, square, rectangular, ovoid, half-sphere/oval, cone, or any other suitable shape). Protrusion 134 and wire 132 may be formed from a single, monolithic material (i.e., protrusion 134 may be formed integrally with wire 132), or protrusion 134 and wire 132 may include separate pieces that are coupled to one another (e.g., via welding, crimping, screw coupling, adhesive, or other suitable mechanisms).

Second segment 104 may include a socket 136 configured to receive control element 130 (e.g., protrusion 134 and/or wire 132). Socket 136 may protrude from an outer surface of another portion of elevator 100, such as from an outer surface of second segment 104. Socket 136 may, in examples, protrude along the B direction from one side of second segment 104. A position of socket 136 depicted in FIGS. 2A-2C is merely exemplary. Socket 136 may, for example, be disposed on an opposite side of elevator 100 (e.g., an opposite side of segment 104 in the B direction). In examples, socket 136 may function as a boss. As shown particularly in FIG. 2A, socket 136 may define a channel 138 for receiving control element 130, which may have an approximately “U” shape. An outer wall 140 of socket 136 may define an opening 142 for receiving an outer surface of protrusion 134. An inner wall 144 of socket 136 may retain protrusion 134 in a desired location within channel 138, rotatably coupled to second segment 104 and unable to move proximally or distally (or along the A or B directions). During assembly, protrusion 134 may be inserted into socket 136 along the B direction, and outer wall 140 of socket 136 may include one or more passages (not shown but which may be disposed, for example, on a distal surface of outer wall 140) for allowing wire 132 to pass through and then be arranged substantially along a longitudinal axis of elevator 100. Other mechanisms may alternatively be used in order to retain protrusion 134 within socket 136.

As shown in FIGS. 2A-2C, socket 136 may be disposed on an external surface of elevator 100 (e.g., of second segment 104), and control element 130 may extend outside of first segment 104 and second segment 102 (i.e., not between walls of elevator 100). In alternatives, control element 130 may extend along an inside of elevator 100 (i.e., between walls of elevator 100). Control element 130 may alternatively be coupled to elevator 100 by any suitable alternative structure (e.g., a control arm coupled to elevator 100).

Inner surfaces of first segment 102 and second segment 104, along with an inner surface of hinge 110, may define a guide surface 160. An accessory device inserted into a working channel of duodenoscope 110 may extend distally from a distal opening of the working channel, and extend on/along guide surface 160. When elevator 100 is raised (e.g., using control element 130), as discussed in further detail below, guide surface 160 may contact the accessory device, exerting a force on the accessory device to articulate it (e.g., a force in a radially outward direction away from a central longitudinal axis of duodenoscope 10). As elevator 100 is lowered, the accessory device may resume an approximately straight configuration (i.e., not be articulated).

Guide surface 160 may have any suitable shape. As best shown in FIG. 2C, along first segment 102 and second segment 104, guide surface 160 may have an approximately “U” shaped cross-section, with a central surface 162 (a lower surface in the coordinate system of FIGS. 2B and 2C) and side surfaces 164 (extending in an up/down direction of FIGS. 2B and 2C). As shown in FIGS. 2A-2C, central surface 162 may be approximately planar, with a plane of central surface 162 being approximately along the proximal/distal directions and the B direction. Side surfaces 164 also may be approximately planar, with their planes being approximately along the A direction and the proximal/distal direction. Central surface 162 and side surfaces 164 may join at a curved junction. At hinge 110, guide surface 160 may include only central surface 162 and may not include side surfaces 164. In operation, central surface 162 may exert forces on an accessory device to articulate it as elevator 100 is raised. Side surfaces 164 may retain the accessory device on central surface 162 (i.e., prevent the accessory device from sliding off elevator 100). However, guide surface 160 may have any suitable shape and is not limited to the configuration described above.

As shown in FIGS. 2A and 2B, proximal edge 172 of segment 102 and distal edge 174 of segment 104 may be tapered. For example, proximal edge 172 may taper inward along the proximal direction. Distal edge 174 may taper inward along a distal direction. As shown in FIG. 2B, a taper of proximal edge 172 may be more gradual than a taper of distal edge 174. However, such a configuration is merely exemplary, and proximal edge 172 and distal edge 174 may have any suitable profile.

As shown in FIGS. 2A-2C, elevator 100 is in an open (i.e., lowered, unarticulated, or unraised) configuration. In the open configuration, as best shown in FIGS. 2A and 2B, elevator 100 is substantially straight, extending in a proximal/distal direction. In particular, central surface 162 is approximately flat between the proximal end of elevator 100 and the distal end of elevator 100 (i.e., in a cross-section of elevator 100 taken along a longitudinal axis of elevator 100, central surface 162 would form an approximately straight line from a proximal end of elevator 100 to a distal end of elevator 100). In other words, all of the segments of elevator 100 extend may along a substantially straight line, which extends parallel to and/or coaxial with a longitudinal axis of elevator 100. Thus, an accessory device received by elevator 100 (e.g., by guide surface 160) in the open configuration may extend approximately parallel to the proximal/distal direction (e.g., approximately parallel to a central longitudinal axis of duodenoscope 10). Thus, the accessory device may extend distally from duodenoscope 10, without extending substantially radially outward/inward. Such a configuration may be desirable for performing medical procedures or some steps of a medical procedure with a forward-facing (i.e., distally-facing tool). In some examples, duodenoscope 10 may include forward-facing (i.e., distally facing) imaging devices (e.g., cameras) and/or lighting elements for performing such forward-facing medical procedures or steps of a medical procedure. Further details of a closed (i.e., raised, articulated) configuration of elevator 100 will be discussed below (e.g., with respect to FIG. 4).

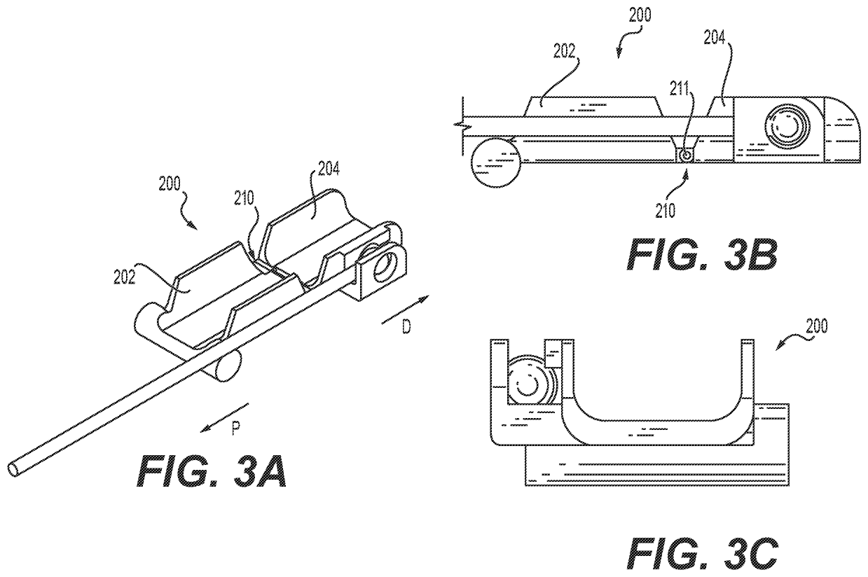

Turning to FIGS. 3A-3C, another exemplary elevator 200 may include any of the features of elevator 100, described above, except as described herein. Where possible, corresponding reference numbers (i.e., reference numbers including the same second and third digits) are used to refer to corresponding structures. Elevator 200 may include a first, proximal segment 202 and a second, distal segment 204. First segment 202 may be a separate, discrete piece/structure from second segment 204. In other words, first segment 202 and second segment 204 may not be monolithically formed from one piece of material. Instead, first segment 202 may be formed from a separate piece of material from second segment 204 or may be formed from a same piece of material and then separated from one another. First segment 202 and second segment 204 may be joined at a hinge 210. For example, one or more pins 211 (FIG. 3B) may extend through portion(s) of first segment 202 and second segment 204 to rotatably connect first segment 202 to second segment 204. In alternatives, other types of fasteners (e.g., rivets, screws) or other types of hinges may be utilized. A distal end of first segment 202 and a proximal end of second segment 204 may be configured so as to allow second segment 204 to articulate relative to first segment 202 (and vice versa). For example, shapes of first segment 202 and second segment 204 may be chosen to accommodate such articulation.

FIG. 4 shows an elevator 300 (having any of the properties of elevators 100, 200) in a partially-closed (i.e., an articulated or partially-raised) configuration. Where possible, corresponding reference numbers (i.e., reference numbers including the same second and third digits) are used to refer to corresponding structures. Elevator 300 may include a first, proximal segment 302 and a second, distal segment 304, joined at a hinge 310. Although first segment 302 and second segment 304 are depicted as separate elements, joined by a hinge 310 similar to hinge 210 of FIGS. 3A-3C, it will be appreciated that hinge 310 may have any of the properties of hinge 110 of FIGS. 2A-2C, and first segment 302 and second segment 304 may be integrally formed.

Axle 320 may be rotatably coupled to distal tip 20 such that it is rotatable about an axis extending into/out of the page of FIG. 4, but is otherwise substantially unable to move relative to distal tip 20. Similarly, a protrusion 334 of a control element 330 may be rotatable relative to a socket 336, but may be otherwise substantially unable to move relative to socket 336.

As shown in FIG. 4, control element 330 may be moved proximally (e.g., via elevator control lever 38 of duodenoscope 10) by an operator. As control element 330 moves proximally, control element 330 may exert a force on second segment 304. Control element 330 may cause second segment 304 to rotate/pivot about hinge 310. Thus, second segment 304 may move relative to first segment 302. As control element 330 is initially moved proximally, first segment 302 may not initially rotate about axle 320. Thus, first segment 302 may continue to lie substantially along a longitudinal axis of distal tip 20 (i.e., along a proximal/distal direction as labeled by the arrows “P” and “D” in FIG. 4).

As shown in FIG. 4, articulation of elevator 300 may bring proximal surface 314 of second segment 304 closer to distal surface 312 of first segment 302. For example, proximal surface 314 of second segment 304 may be approximately parallel to distal surface 312 of first segment 302 and/or contacting distal surface 312 of first segment 302. Shapes of proximal surface 314 and distal surface 312 (e.g., angles of tapers of proximal surface 314 and distal surface 312) may define an articulation angle of elevator 300, as described in further detail with respect to FIG. 5, below. Alternatively, other structures of elevator 300 (e.g., stops, protrusions, or features of hinge 310) may define an extent to which second segment 304 articulates with respect to first segment 302.

As control element 330 continues to be moved proximally, first segment 302 may rotate about axle 320 (not shown in FIG. 4). For example, first segment 302 may rotate about axle 320 after second segment 304 is fully articulated with respect to first segment 302 (e.g., after no further bending about hinge 310 is possible because, for example, distal surface 312 abuts proximal surface 314). As first segment 302 rotates about axle 320, a distal end 303 of first segment 302 moves in an upward direction of FIG. 4/rotates in a counter-clockwise direction of FIG. 4 about axle 320. In other words, as first segment 302 rotates, first segment 302 may become transverse to a longitudinal axis of distal tip 20. Features of elevator 300, control element 330, elevator control lever 38, or other features of duodenoscope 10) may limit a scope of rotation of first segment 302 about axle 320, defining a range of motion of elevator 300.

As elevator 300 is articulated (i.e., opened or closed), an accessory device (not shown) may be articulated, as described above with respect to FIGS. 2A-2C. In the partially-articulated configuration of FIG. 4, the accessory device may be articulated, such that a distal portion of the accessory device has a first angle with respect to a more proximal portion of the accessory device. In a fully articulated configuration (in which first segment 302 has rotated about axle 320), the distal portion of the accessory device may have a second angle with respect to the proximal portion of the accessory device. The second angle may be smaller than the first angle, such that the first angle is more open than the second angle. In other words, the accessory device may be more articulated/bent in the fully closed configuration than in the partially closed configuration of elevator 300.

In a partially or fully articulated configuration of elevator 300, an operator may perform medical procedures, such as cannulating a papilla of a subject. In some examples, duodenoscope 10 may include side-facing (i.e., radially outward-facing) imaging devices (e.g., cameras) and/or lighting elements for performing such medical procedures or steps of a medical procedure.

Elevator 300 may be moved in a reverse direction (to move elevator 300 from a closed position to an open position) by moving control element 330 distally. Movement may occur in the reverse order to that described above, with elevator 300 first rotating about axle 320 (assuming elevator 300 has previously been rotated about axle 320 to close elevator 300) and then with second segment 304 rotating with respect to first segment 302, until elevator 300 is lying flat/fully open.

FIG. 5 shows an elevator 400 (having any of the properties of elevators 100, 200, 300) in an open configuration. Where possible, corresponding reference numbers (i.e., reference numbers including the same second and third digits) are used to refer to corresponding structures. Elevator 400 may include a first, proximal segment 402 and a second, distal segment 404, joined at a hinge 410. Although first segment 402 and second segment 404 are depicted as being integrally formed with one another, joined by a hinge 410 similar to hinge 110 of FIGS. 2A-2C, it will be appreciated that hinge 410 may have any of the properties of hinge 210 of FIGS. 3A-3C, and first segment 402 and second segment 404 may be separate structures from one another.

As shown in FIG. 5, distal surface 412 of first segment 402 may taper toward hinge 410 in a distal direction, and a proximal surface 414 of second segment 404 may taper toward hinge 410 in a proximal direction. An axis Y₁ is shown in FIG. 5 bisecting hinge 410 and extending perpendicularly to a longitudinal axis of elevator 400 (i.e., perpendicularly to the proximal and distal directions), in an up/down direction of FIG. 5. Each of distal surface 412 and proximal surface 414 may have an angle θ (having any suitable value) with respect to axis Y₁. Angle θ may be chosen based on a desired final profile (e.g., curve) of elevator 400 and a number of segments of elevator 400. Angle θ may be chosen to produce different accessory device angles seen via imaging device 22 when elevator 400 is locked, for example an accessory device angle configured to position the accessory device within a field of view of imaging device 22. As discussed above, angles of distal surface 412 and proximal surface 414 may define an articulation angle of elevator 400. For example, elevator 400 may be able to bend an angle of approximately 2θ (i.e., second segment 404 may be able to bend an angle of 2θ with respect to first segment 402), until distal surface 412 and proximal surface 414 are abutting or near to one another (e.g., as shown in FIG. 4 as to distal surface 312 and proximal surface 314 of elevator 300).

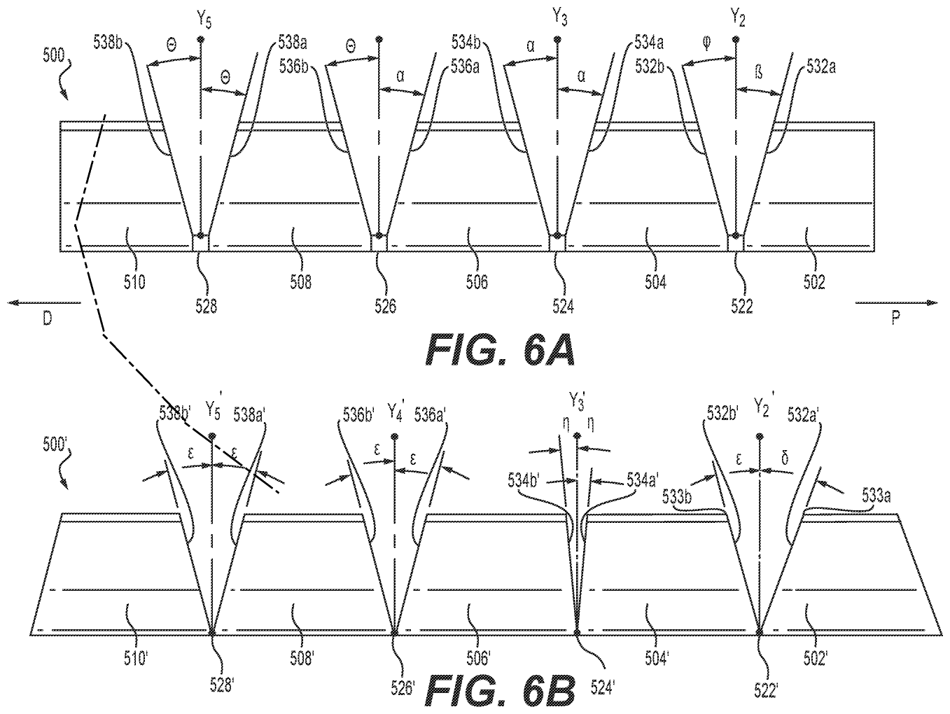

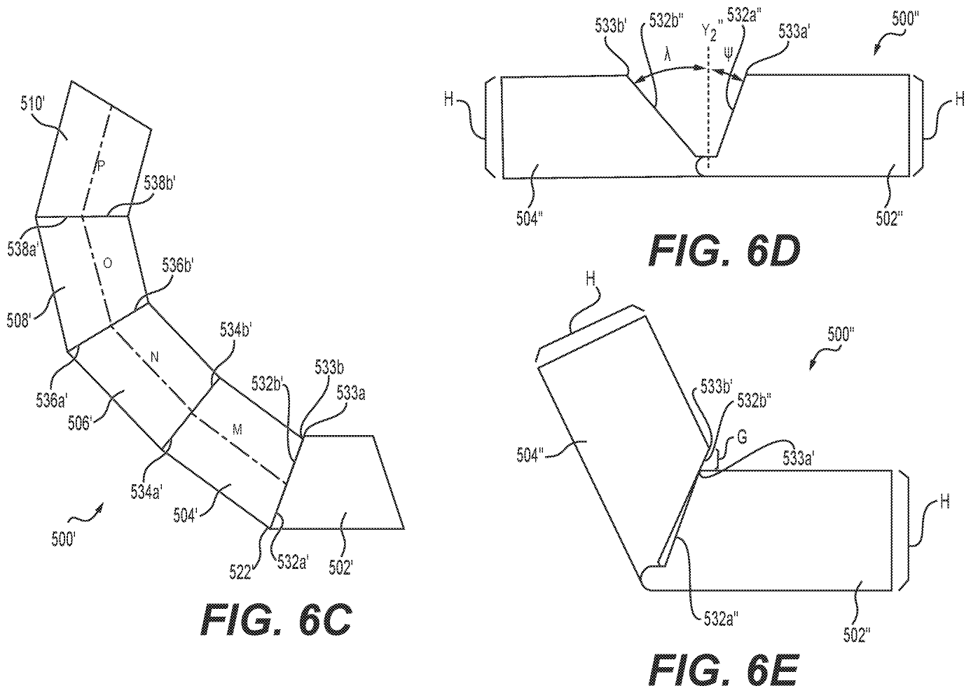

FIG. 6A shows an alternative elevator 500, which may have any of the properties of elevators 100, 200, 300, 400, except as specified herein. Elevator 500 may include a plurality of segments 502, 504, 506, 508, 510. Segments 502, 504, 506, 508, 510 may have any of the properties of any of the segments described above. Segments 502 and 504 may be coupled at a hinge 522, segments 504 and 506 may be coupled at a hinge 524, segments 506 and 508 may be coupled at a hinge 526, and segments 508 and 510 may be coupled at a hinge 528. Hinges 522, 524, 526, 528 may have any of the properties of the hinges described above. Some or all of segments 502, 504, 506, 508, 510 may be monolithically formed, with some or all of hinges 522, 524, 526, 528 including living hinges (like, for example, hinge 110). Additionally or alternatively, some or all of segments 502, 504, 506, 508, 510 may be separately formed structures/pieces, with some or all of hinges 522, 524, 526, 528 joining the separate structures (like, for example, hinge 210). A combination of monolithically formed structures and separate segment pieces may be utilized in elevator 500.

Axes Y₂, Y₃, Y₄, Y₅ are depicted in FIG. 6 as extending through respective midpoints of hinges 522, 524, 526, 528, perpendicularly to a longitudinal axis of elevator 500 (i.e., in an up/down direction of FIG. 6). Axes Y₂, Y₃, Y₄, Y₅ may be substantially parallel to one another. Segment 502 may have a distal surface 532a, and segment 504 may have a proximal surface 532b. Segment 504 may have a distal surface 534a, and segment 506 may have a proximal surface 534b. Segment 506 may have a distal surface 536a, and segment 508 may have a proximal surface 536b. Segment 508 may have a distal surface 538a, and segment 510 may have a proximal surface 538b. Surfaces 532a, 532b may be on either side of hinge 522, surfaces 534a, 534b may be on either side of hinge 524, surfaces 536a, 536b may be on either side of hinge 526, and surfaces 538a, 538b may be on either side of hinge 528.

As described below, the surfaces of segments 502, 504, 506, 508, 510 may have varying angles with respect to axes Y₂, Y₃, Y₄, Y₅. These varied angles may provide for different articulation profiles of elevator 500. For example, some of 502, 504, 506, 508, 510 may bend at sharper angles to one another than others of 502, 504, 506, 508, 510. The angle combinations described herein are merely exemplary; any suitable combination of angles may be chosen to achieve a desired articulation profile of elevator 500.

Distal surface 532a may have an angle β with respect to axis Y₂. Proximal surface 532b may have an angle ϕ with respect to axis Y₂. In some examples, angle β may differ from angle ϕ. The angles β and ϕ may be chosen so as to provide a desired articulation. Distal surface 534a and proximal surface 534b each may have an angle α with respect to axis Y₅. Distal surface 536a may have an angle α with respect to axis Y₄. In some examples, angle α may differ from angles β and ϕ. Proximal surface 536b may have an angle θ with respect to axis Y₄. Angle θ may differ from angles α, β, and ϕ. Distal surface 538a and proximal surface 538b each may have an angle θ with respect to axis Y₅.

The arrangements and angles described above are merely exemplary. Angles of surfaces about hinges 522, 524, 526, 528 may be chosen so as to achieve a variety of articulation profiles. For example, hinges 522, 524, 526, 528 may have varied angles at which adjacent links are bent with respect to one another. In alternatives, hinges 522, 524, 526, 528 each may bend at the same angles. In addition to having end surfaces with varied angles, segments 502, 504, 506, 508, 510 may have the same or different lengths (along a longitudinal axis of elevator 500, i.e., along proximal/distal directions). Lengths of segments 502, 504, 506, 508, 510 may be chosen so as to give elevator 500 a desired shape/articulation profile when in an articulated (e.g., closed or partially closed) configuration.

FIGS. 6B and 6C show an elevator 500′ having any of the properties of elevator 500, except as described herein. Reference numbers of elevator 500′ may correspond to reference numbers of elevator 500, with a “prime” added to the end of the reference numbers of elevator 500. Corresponding reference numbers may have corresponding properties, except as explained below.

As shown in FIG. 6B, distal surface 532a′ may have an angle δ with respect to axis Y₂′. Proximal surface 532b′ may have an angle ε with respect to axis Y₂′. In some examples, angle δ may differ from angle E. The angles δ and ε may be chosen so as to provide a desired articulation. As shown in FIG. 6C and discussed with respect to FIGS. 6D-6E below, because angles δ and ε differ from one another (e.g., because angle δ is larger than angle ε), when elevator 500′ is articulated, a corner 533a of distal surface 532a′ may extend slightly above (in the reference frame of FIGS. 6B and 6C) a corner 533b of proximal surface 532b′. Such a configuration may ensure that elevator 500′ does not contact a housing or other portion of distal tip 20 in an undesired location or may provide other mechanical advantages. Distal surface 534a′ and proximal surface 534b′ each may have an angle r with respect to axis Y₃′. Distal surface 536a′ and proximal surface 534b′ each may have an angle ε with respect to axis Y₄′. Distal surface 538a′ and proximal surface 538b′ each may have an angle ε with respect to axis Y₅′. Angles δ, ε, and η may each differ from one another.

As shown in FIG. 6C, the relative angles between the surfaces of elevator 500′ may define different articulation profiles along elevator 500′. For example, segments 504′ and 506′ may be less angled with respect to one another than segments 506′ and 508,′ due to a smaller size of angle r relative to angle E. In other words, an angle between longitudinal axis M along segment 504′ and a longitudinal axis N along segment 506′ may be closer to 180 degrees than an angle between longitudinal axis N and a longitudinal axis O along segment 508′. Because the angles between segments 508′ and 510′ are the same as the angles between segments 506′ and 508′, an angle between longitudinal axis O along segment 508′ and longitudinal axis P along segment 510′ may be the same.

FIGS. 6D and 6E depict another elevator 500″ having any of the properties of elevator 500, 500′ except as described herein. Reference numbers of elevator 500″ may correspond to reference numbers of elevator 500, with a double “prime” added to the end of the reference numbers of elevator 500. Corresponding reference numbers may have corresponding properties, except as explained below. Although elevator 500″ is depicted as having only two segments 502″, 504″, it will be appreciated that elevator 500″ may have any suitable number of segments.

As shown in FIG. 6D, distal surface 532a″ may have an angle ψ with respect to axis Y₂″. Proximal surface 532b″ may have an angle λ with respect to axis Y₂″. Angle ψ may differ from angle λ. The angles ψ and λ may be chosen so as to provide a desired articulation. As shown in FIG. 6D, in a relaxed configuration, a height H of segments 502″ and 504″ (in an up/down direction of FIG. 6D) may be the same. As shown in FIG. 6E, because angles ψ and λ differ from one another (e.g., because angle λ is larger than angle ψ), when elevator 500″ is articulated, a corner 533a′ of distal surface 532a″ may extend above (in the reference frame of FIG. 6E) a corner 533b′ of proximal surface 532b″, such that a gap G is formed between corners 533a′ and 533b′. Furthermore, as shown in FIG. 6E, surfaces 532a″ and 532b″ may not be flush against one another, such that a gap is formed therebetween.

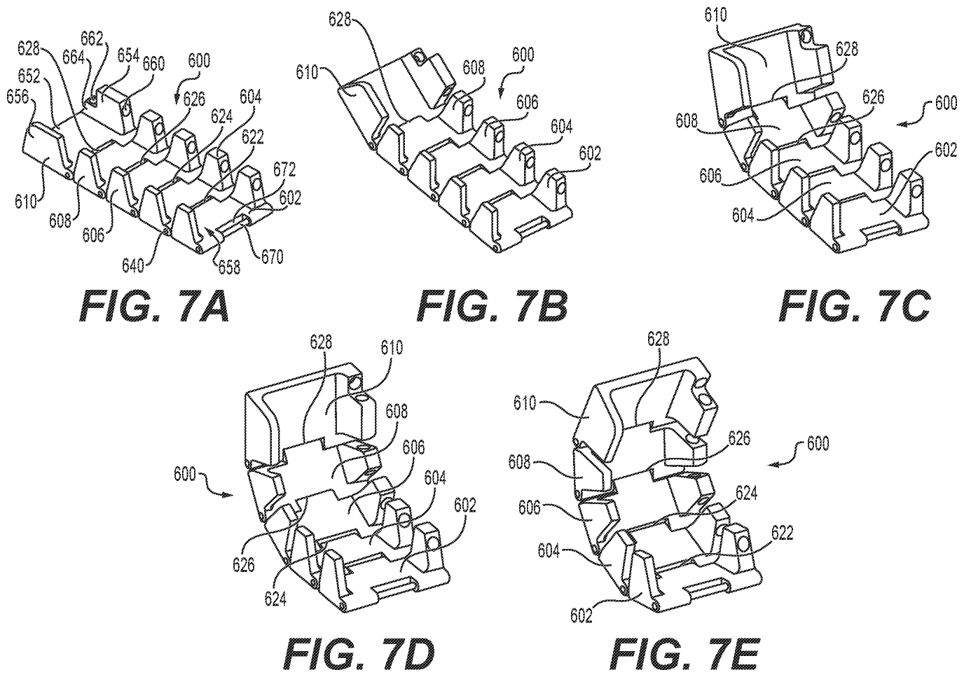

FIGS. 7A-7E depict an elevator 600 in varying stages of articulation, from fully open (i.e., unarticulated, un-raised, or lowered) in FIG. 7A to almost fully closed (i.e., fully articulated or raised) in FIGS. 7B-7D and fully closed in FIG. 7E. Elevator 600 may have any of the features of elevators 100, 200, 300, 400, 500 described above and may be used in conjunction with duodenoscope 10. Corresponding reference numbers to elevator 500 (i.e., reference numbers having the same second and third digits) are used where feasible.

Segments 602, 604, 606, 608, 610 may be connected via hinges 622, 624, 626, 628. Segment 602 may be a proximal-most segment, and segment 610 may be a distal-most segment. Although five segments are depicted, it will be appreciated that any suitable number of segments may be utilized (e.g., two, three, four, six, seven, or more segments). Although FIGS. 7A-7E depict segments 602, 604, 606, 608, 610 as being separate structures/pieces, it will be appreciated that segments 602, 604, 606, 608, 610 alternatively may be formed from a single, monolithic piece of material. Similarly, although hinges 622, 624, 626, 628 are illustrated as including pins 640 (labelled with respect to hinge 622 on FIG. 7A and also present on hinges 624, 626, 628), it will be appreciated that hinges 622, 624, 626, 628 may use any alternative connecting structure and/or may include living hinges.

Each of segments 602, 604, 606, 608, 610 may have a base wall 652, a first side wall 654, and a second side wall 656 (labeled with respect to segment 610 on FIG. 7A and also present on segments 602, 604, 606, 608). Base wall 652 and side walls 654, 656 may define a guide surface 658, for contacting and guiding an accessory device. Side walls 654 each may define a channel 660, extending approximately parallel to a longitudinal axis of elevator 600/distal tip 20 of duodenoscope 10. A control element (not shown in FIGS. 7A-7E) having any of the properties of the control elements discussed above may extend through channels 660 of each of side walls 654. Control element may be movable relative to channels 660. Side walls 654 may have a greater thickness (along an axis extending between side wall 654 and side wall 656 of a given segment) than side walls 656, in order to accommodate channels 660.

Segment 610 (or any distal-most segment) may include a fixation point 662 for fixing a distal tip of the control element to segment 610. For example, the control element may be fixed to fixation point 662 via, e.g., a friction fit, adhesive, crimping, welding, fasteners, or any other suitable mechanism. Fixation point 662 may be disposed on a protrusion 664 of side wall 654 of segment 610. Protrusion 664 may protrude distally from another portion of side wall 654. Segment 610 may be longer, in a proximal/distal direction (i.e., along the longitudinal axis of elevator 600) in order to accommodate protrusion 664. Side wall 656 of segment 600 (on an opposite side of segment 610 from side wall 654) may have a length that corresponds to a length of side wall 654 of segment 610, including protrusion 664.

Segment 602 (or any proximal-most segment) may include an axle 670. In some examples, axle 670 may be formed integrally with segment 602, from a monolithic piece of material. In other examples, axle 670 may be a separate component that is affixed to segment 602. Axle 670 may have any suitable configuration. For example, base wall 652 of segment 602 may define a notch 672, and axle 670 may extend across the notch. Axle 670 may extend approximately perpendicularly to a longitudinal axis of elevator 600, along a direction extending between side walls 654, 656 of segment 602. Axle 670 may be received by an element of distal tip 20 of duodenoscope 10. For example, distal tip 20 may include a hole or recess that receives axle 670. Segment 602 may be rotatable relative to distal tip 20 about axle 670. In alternatives, axle 670 may be removed, and segment 602 may be fixed/may be non-rotatable relative to distal tip 20.

In the configuration of FIG. 7A, elevator 600 may be in a fully open configuration. A portion of guiding surface 658 defined by base wall 652 may be approximately planar/flat, including along a proximal/distal direction. In other words, segments 602, 604, 606, 608, 610 may be aligned so that they are substantially straight. An accessory tool inserted into a working channel of duodenoscope 10 may extend along guide surface 658 in a generally unarticulated configuration, in which the accessory tool extends approximately along a longitudinal axis of duodenoscope 10/distal tip 20, extending from distal tip 20.

In the configuration of FIG. 7B, distal segment 610 may be rotated/pivoted with respect to distal segment 608, due to proximal movement of the control member. The control member may pull on segment 610, causing it to pivot/rotate with respect to segment 608, without moving segments 602, 604, 606, 608. In the configuration of FIG. 7C, the control member may have been moved further proximally, as compared to the configuration of FIG. 7B. The rotated/pivoted relationship between segments 608 and 610 at hinge 628 may be retained, and segment 608 may be rotated/pivoted with respect to segment 606 at hinge 626, without moving segments 602, 604, 606. In the configuration of FIG. 7D, the control member may have been moved further proximally, as compared to the configuration of FIG. 7C. The rotated/pivoted relationship between segments 608 and 610 at hinge 628 and segments 606 and 608 at hinge 626 may be retained, and segment 606 may be rotated/pivoted with respect to segment 604 at hinge 624, without moving segments 602, 604. In the configuration of FIG. 7E, the control member may have been moved further proximally, as compared to the configuration of FIG. 7D. The rotated/pivoted relationship between segments 608 and 610 at hinge 628, segments 606 and 608 at hinge 626, and segments 604 and 606 at hinge 624 may be retained, and segment 604 may be rotated/pivoted with respect to segment 602 at hinge 622, without moving segment 602. In a further configuration, not shown, the control member may be moved further proximally, as compared to the configuration of FIG. 7D, in order to rotate segment 602 about axle 670. As the control wire is moved proximally, elevator 600 may lower in the reverse order to that described above.

Handle 12 of duodenoscope 10 may include features (e.g., markings or other visual feedback, or tactile feedback) for incrementally adjusting elevator control lever 38, to allow for articulation to pre-determined configuration(s) such as those shown in FIG. 7A-7E. Handle 12 of duodenoscope 10 may additionally or alternatively include locking/retaining features for locking elevator control lever 38 in a desired position, such as positions corresponding to one or more of the configurations of FIGS. 7A-7E.

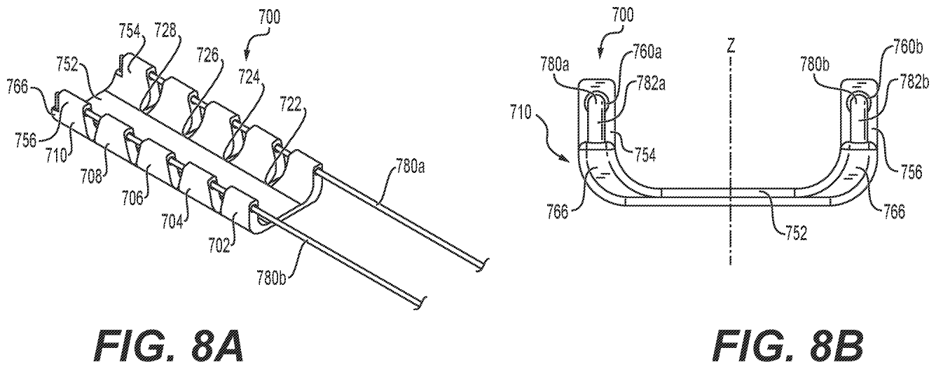

FIGS. 8A and 8B show another exemplary elevator 700, having any of the features of elevators 100, 200, 300, 400, 500, 600, described above, and usable with duodenoscope 10. FIG. 8A shows a perspective view of elevator 700, and FIG. 8B shows a proximally facing view of a distal end of elevator 700. Corresponding reference numbers to elevators 500, 600 (i.e., reference numbers having the same second and third digits) are used where feasible. Elevator 700 may include a plurality of segments 702, 704, 706, 708, 710 joined by hinges 722, 724, 726, 728. As shown in FIG. 8A, segments 702, 704, 706, 708, 710 may be formed of a single, integral piece of material, with hinges 722, 724, 726, 728 including living hinges. In alternatives, 702, 704, 706, 708, 710 may be formed of separate pieces, coupled together at hinges 722, 724, 726, 728. Any suitable type of hinging structure may be utilized for hinges 722, 724, 726, 728. As above, a number of segments/hinges depicted is merely exemplary, and any suitable number of segments/hinges may be utilized.

Each of segments 702, 704, 706, 708, 710 may have a base wall 752, a first side wall 754, and a second side wall 756 (labeled with respect to segment 710 on FIG. 8A and also present on segments 702, 704, 706, 708). Side walls 754 each may define a channel 760a (see FIG. 8B), extending approximately parallel to a longitudinal axis of elevator 700/distal tip 20 of duodenoscope 10 (i.e., into/out of the page of FIG. 8B). Side walls 756 each may define a channel 760b (see FIG. 8B), extending approximately parallel to a longitudinal axis of elevator 700/distal tip 20 of duodenoscope 10 (i.e., into/out of the page of FIG. 8B). Channels 760a, 760b may have any of the properties of channels 660 of elevator 600, described above.

A first control element 780a (having any of the features of the control elements discussed above) may extend through channels 760a of each of segments 702, 704, 706, 708, 710. A second control element 780b may extend through channels 760b of each of segments 702, 704, 706, 708, 710. Distal end 782a of first control element 780a and distal end 782b of second control element 780b may each be coupled to a distal face (or other portion) of segment 710 (i.e., a distalmost segment). For example, distal ends 782a, 782b may extend transversely to more proximal portions of first control element 780a and second control element 780b, respectively (e.g., at approximately a 90 degree angle to more proximal portions first control element 780a and second control element 780b, respectively). Channels 760a, 760b may be located near an edge of side walls 754, 756, respectively, that is farthest from base wall 752. Distal ends 782a, 782b may extend toward base wall 752 and be fixed to side walls 754, 756, respectively, of segment 710. For example, each of side walls 754, 756 may include a protrusion 766, which extends distally from a more proximal portion of side walls 754, 756, respectively. Distal ends 782a, 782b may be coupled to protrusions 766. In an alternative, control elements 780a, 780b may form a loop (i.e., be a single continuous piece), about or through segment 710. For example, distal ends 782a, 782b may be coupled to one another, extending through side walls 754, 756 and base wall 752 of segment 710.

As shown in FIG. 8B, elevator 700 may be symmetrical about a plane Z (i.e., a plane defined by a longitudinal axis of elevator 700 and an axis bisecting base wall 752, between side walls 754, 756). Control elements 780a, 780b may be coupled to the same elevator control lever 38. For example, proximal ends of 780a, 780b may be coupled to elevator control lever 38 and/or to one another or a single control element (not shown) within shaft 18 or handle 12. Proximal or distal movement of control elements 780a, 780b may cause elevator 700 to close or open, respectively. Use of two control elements 780a, 780b may decrease a force required to be exerted on elevator control lever 38 and may produce a more even force on elevator 700. Alternatively, control elements 780a, 780b may be coupled to different actuators for independent articulation or to a joystick-type actuator. In such a configuration, movement of one of control elements 780a, 780b relative to the other of control elements 780a, 780b may allow for bending of elevator 700 in directions transverse to a longitudinal axis of elevator 700 (e.g., side to side articulation like a distal end of an articulable shaft of a medical device such as a duodenoscope or other type of scope) or rotation of elevator 700.

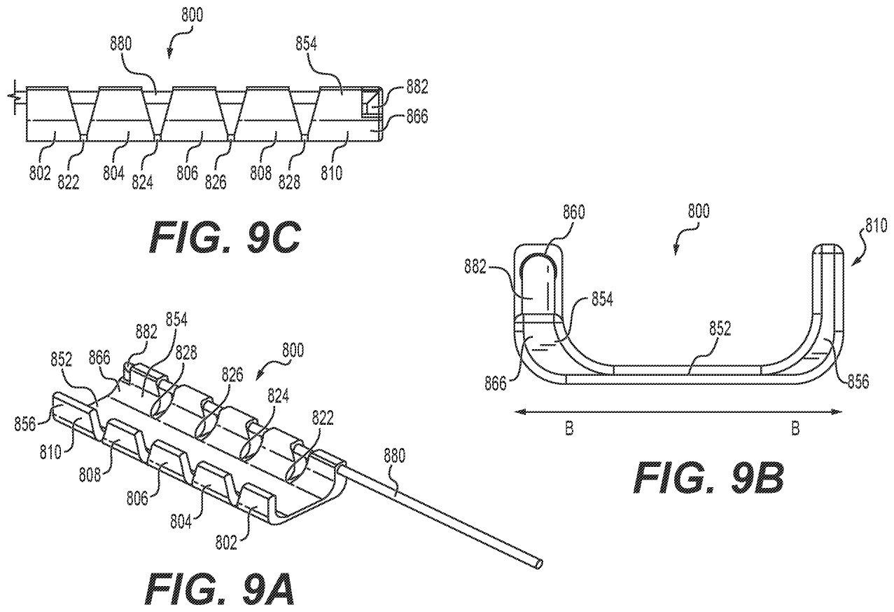

FIGS. 9A-9C depict an alternative elevator 800, which may have any of the properties of elevators 100, 200, 300, 400, 500, 600, 700, except as specified herein. FIG. 9A depicts a perspective view of elevator 800, FIG. 9B is a proximal-facing view of a distal end of elevator 800, and FIG. 9C is a side view of elevator 800. Corresponding reference numbers to elevators 500, 600, 700 (i.e., reference numbers having the same second and third digits) are used where feasible. Elevator 800 may include a plurality of segments 802, 804, 806, 808, 810 joined by hinges 822, 824, 826, 828. As shown in FIG. 9A, segments 802, 804, 806, 808, 810 may be formed of a single, integral piece of material, with hinges 822, 824, 826, 828 including living hinges. In alternatives, 802, 804, 806, 808, 810 may be formed of separate pieces, coupled together at hinges 822, 824, 826, 828. Any suitable type of hinging structure may be utilized for hinges 822, 824, 826, 828. As discussed above, a number of segments/hinges depicted is merely exemplary, and any suitable number of segments/hinges may be utilized.

Each of segments 802, 804, 806, 808, 810 may have a base wall 852, a first side wall 854, and a second side wall 856 (labeled with respect to segment 810 on FIG. 8A and also present on segments 802, 804, 806, 808). Side walls 854 each may define a channel 860 (see FIG. 8B), extending approximately parallel to a longitudinal axis of elevator 800/distal tip 80 of duodenoscope 10 (i.e., into/out of the page of FIG. 9B). In contrast to elevator 700, side walls 856 may not define such channels.

A control element 880 (having any of the features of the control elements discussed above) may extend through channels 860 of each of segments 802, 804, 806, 808, 810. A distal end 882 of control element 880 may be coupled to a distal face (or other portion) of segment 810 (i.e., a distalmost segment). For example, distal end 882 may extend transversely to more proximal portions of control element 880 (e.g., at approximately a 90 degree angle to control element 880. Distal end 882 may extend toward base wall 852 and be fixed to side walls 854 of segment 810. For example, side wall 854 may include a protrusion 866 that extends distally from a more proximal portion of side wall 854. Distal ends 882 may be coupled to protrusion 866.

As compared to side wall 856, side wall 854 may be wider in a direction B (see FIG. 9B), which is perpendicular to a longitudinal axis of elevator 800, extending between side wall 854 and side wall 856. A wider width of side wall 854 than side wall 856 may be due to a width of side wall 854 required to define channel 860. For side wall 856, it may be desirable to have a smaller wall width in order to save space in distal tip 20. It will be appreciated that an orientation of side walls 856, 854, is merely exemplary, and side walls 856, 854 may be reversed with one another.

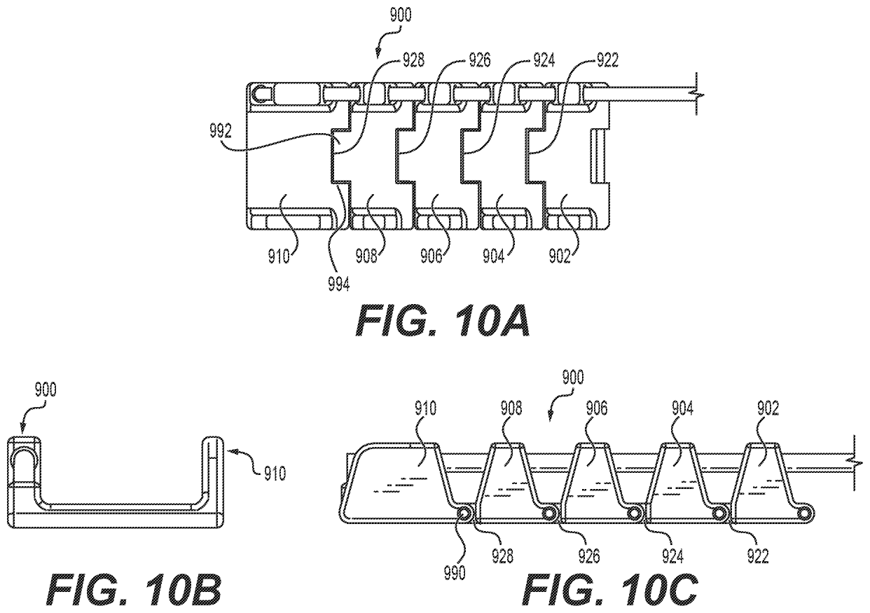

FIGS. 10A-10C depict an alternative elevator 900, which may have any of the properties of elevators 100, 200, 300, 400, 500, 600, 700, 800 except as specified herein. FIG. 10A depicts a top view of elevator 900, FIG. 10B is a proximal-facing view of a distal end of elevator 900, and FIG. 10C is a side view of elevator 900. Corresponding reference numbers to elevators 500, 600, 700, 800 (i.e., reference numbers having the same second and third digits) are used where feasible. Elevator 900 may include a plurality of segments 902, 904, 906, 908, 910 joined by hinges 922, 924, 926, 928. As discussed above, a number of segments/hinges depicted is merely exemplary, and any suitable number of segments/hinges may be utilized.

As shown in FIG. 9A, segments 902, 904, 906, 908, 910 may be formed of separate pieces, coupled together at hinges 922, 924, 926, 928. Any suitable type of hinging structure may be utilized for hinges 922, 924, 926, 928. For example, as shown in FIG. 9C, hinges 922, 924, 926, 928 may include pins 990, which may extend through all or part of a width (a direction into/out of the page of FIG. 9C), of hinges 922, 924, 926, 928. For example, pins 990 may extend through openings of 902, 904, 906, 908, 910 in order to form 922, 924, 926, 928. Each of segments 902, 904, 906, 908 may include a protrusion 992 (FIG. 10A), which may extend from a distal side of segments 902, 904, 906, 908. Protrusion 992 may have, for example, a rectangular shape. Each of segments 910, 908, 906, 905 may include a corresponding notch 994 (FIG. 10A, formed in a proximal side of 910, 908, 906, 905. Notch 994 may have, for example, a rectangular shape. Notch 994 and protrusion 992 may have complementary shapes, such that protrusion 992 may fit within notch 994. Protrusion 992 may be able to rotate relative to notch 994, about pin 990. For example, a pin 990 may extend through an outer wall of a segment (e.g., segment 910), through an opening formed in a side wall of notch 994, through protrusion 992 of a proximally adjacent segment (e.g., segment 908), and through an opening formed on the other side wall of notch 994. The segments 910, 908 are referred to merely for exemplary purposes, and the same principle applies to other segments. In alternatives, a protrusion may extend proximally from a segment (e.g., segment 910) of elevator 900, and a proximally adjacent segment (e.g., segment 908) may include a corresponding notch for receiving the protrusion.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented embodiments can be combined to achieve a same or similar result as one or more of the disclosed embodiments. Accordingly, the invention is not to be considered as limited by the foregoing description.

We claim:

1. An elevator of a medical device, comprising:
a plurality of segments joined together by at least one hinge, wherein a first segment of the plurality of segments is a discrete structure; and
a control element coupled to at least one segment of the plurality of segments, wherein proximal or distal movement of the control element is configured to move the at least one segment of the plurality of segments relative to another of the plurality of segments.

2. The elevator of claim 1, wherein all of the plurality of segments are discrete structures.

3. The elevator of claim 1, wherein the plurality of segments consists of the first segment and a second segment.

4. The elevator of claim 1, wherein, in a first configuration, all of the plurality of segments lie along an approximately straight line.

5. The elevator of claim 4, wherein the approximately straight line is approximately parallel to a longitudinal axis of the medical device.

6. The elevator of claim 1, wherein the control element is a first control element, the elevator further comprising a second control element coupled to the at least one segment of the plurality of segments, wherein proximal or distal movement of the second control element is configured to move the at least one segment of the plurality of segments relative to another of the plurality of segments.

7. The elevator of claim 6, wherein each of the plurality of segments defines a first channel for receiving the first control element, and wherein each of the plurality of segments defines a second channel for receiving the second control element.

8. The elevator of claim 6, wherein the first control element is coupled to a first actuator of a handle of the medical device, and wherein the second control element is coupled to a second actuator of a handle of the medical device, wherein the first actuator is separately actuatable from the second actuator.

9. The elevator of claim 1, wherein a distalmost segment of the plurality of segments includes a socket on an outer surface of the distalmost segment, wherein the socket is configured to receive a distal end of the control element.

10. The elevator of claim 1, wherein the first segment includes a first distal surface, wherein a second segment of the plurality of segments includes a first proximal surface and a second distal surface, wherein a third segment of the plurality of segments includes a second proximal surface, wherein the first distal surface of the first segment and the first proximal surface of the second segment are separated by a first angle, wherein the second distal surface of the second segment and the second proximal surface of the third segment are separated by a second angle, and wherein the second angle differs from the first angle.

11. The elevator of claim 1, wherein each segment of the plurality of segments includes a base wall, a first side wall, and a second side wall, wherein the first side wall of each segment defines a channel configured to receive the control element, and wherein a width of the first side wall of each segment is greater than a width of the second side wall of each segment.

12. The elevator of claim 1, wherein the at least one hinge includes a pin or rivet.

13. The elevator of claim 1, wherein a distalmost segment of the plurality of segments includes a distally extending protrusion, wherein the control element is affixed to the protrusion.

14. The elevator of claim 1, wherein a proximalmost segment of the plurality of segments includes an axle configured to be rotatably coupled to a distal tip of the medical device.

15. The elevator of claim 14, wherein at least one of the plurality of segments is configured to rotate, while the control element moves distally, relative to the proximalmost segment of the plurality of segments before the proximalmost segment of the plurality of segments rotates with respect to the distal tip of the medical device.

16. An elevator of a medical device, comprising:
a plurality of segments joined together by at least one hinge;
a first control element coupled to at least one segment of the plurality of segments, wherein proximal or distal movement of the first control element is configured to move the at least one segment of the plurality of segments relative to another of the plurality of segments; and
a second control element coupled to the at least one segment of the plurality of segments, wherein proximal or distal movement of the second control element is configured to move the at least one segment of the plurality of segments relative to another of the plurality of segments.

17. The elevator of claim 16, wherein at least two segments of the plurality of segments are formed monolithically with one another.

18. The elevator of claim 16, wherein each of the plurality of segments defines a first channel for receiving the first control element, and wherein each of the plurality of segments defines a second channel for receiving the second control element.

19. An elevator of a medical device, comprising:
a first segment;
a second segment;
a third segment;
a first hinge between the first segment and the second segment; and
a second hinge between the second segment and the third segment, wherein the first segment includes a first distal surface, wherein the second segment includes a first proximal surface and a second distal surface, wherein the third segment includes a second proximal surface, wherein the first distal surface of the first segment and the first proximal surface of the second segment are separated by a first angle, wherein the second distal surface of the second segment and the second proximal surface of the third segment are separated by a second angle, and wherein the second angle differs from the first angle.

20. The elevator of claim 19, wherein the first segment, the second segment, and the third segment are formed monolithically with one another.
